(19) United States
(12) Patent Application Publication
(10) Pub. No.: US 2001/0047151 A1
(43) Pub. Date: Nov. 29, 2001

(54) Depth controller for epidural needle

(76) Inventors: Yongming Xian, Brooklyn, NY (US); Deye Wei, Brooklyn, NY (US)

Correspondence Address: Yongming Xian, or/and Deye Wei, 72 Bay 37th. Street, 2F, Brooklyn, NY, 11214, US

(21) Appl. No.: 09/773,444

(22) Filed: Feb. 1, 2001

Related U.S. Application Data
(60) Provisional application No. 60/179,598, filed on Feb. 2, 2000.

(51) Int. Cl. A61M 5/00; A61M 5/315

(52) U.S. Cl. 604/117; 604/207; 604/224

(57) ABSTRACT

A method for holding an epidural needle together with a syringe and controlling the depth and advancement of penetration of the epidural needle during the epidural needle insertion into the human epidural space through the multiple layers of the tissues with the apparatus of the invention.

An apparatus for holding an epidural needle together with a syringe and controlling the depth and the advancement of penetration of the epidural needle during the epidural needle insertion into the human epidural space through the multiple layers of the human tissues with the epidural needle held in a slot of an epidural needle holder which is a plate connected to a sliding plate with an angle, and with the epidural needle's wing and the attached syringe to slide and advance on the said sliding plate, on which there is a screw with a screw nut, which can move forwards and backwards to stop further advancement of the epidural needle together with the syringe by blocking the wing of the epidural needle together with the epidural needle and the attached syringe to slide and advance further over the said sliding plate. The said screw nut can be turned and driven half to one millimeter further toward the patient to allow the epidural needle to advance half to one millimeter deeper in each attempt until entering the human epidural space with multiple attempts, which is identified and confirmed with the lose of resistance technique. A scale in centimeter marked on each side of the said sliding plate indicates directly how many centimeters deep the tip of the said epidural needle has been inserted from the skin.

### BACKGROUND OF THE INVENTION

1. Field of the Invention

The invention relates to an apparatus and a method for controlling the depth and the advancement of an epidural needle during insertion of the epidural needle into the human epidural space for epidual anesthesia or drug administration.

2. Background of the Invention

For epidural anesthesia and epidural drug administration, an epidural needle is inserted slowly and manually on the patient's back through the skin, subcutaneous tissue, supraspinous ligament, interspinous ligament, ligamentum flavum, and then into the epidural space. The position of the tip of epidural needle in the epidural space is identified and confirmed with the loss of resistance technique or the “hanging-drop” technique. Once the tip of the epidural needle in the epidural space is confirmed, an epidural catheter may be placed or a drug may be administered into the epidural space through the epidural needle. The epidural needle is then removed. Drug may be administered through the catheter into the epidural space intermittently or continuously. In current literature and practice, the epidural needle is held by one hand or two hands during the insertion. There are two ways to hold the epidural needle: one-hand technique and two-hand technique. In one hand technique, the thumb and the middle finger hold the epidural needle or the hub of the epidural needle, with the index finger held against the patient's back, or the fingers hold the epidural needle with the dorsum of the hand held against the patient's back. In two-hand technique, the thumbs and the index fingers of the two hands hold the wing of the epidural needle, with the middle fingers held against the patient's back.

Since the epidural space is small, a sudden and too much advancement of the epidural needle can pass through the epidural space, penetrate the dura mater and cause dural puncture (wet tap), a complication needed avoidance. With the above two mentioned hand-hold techniques, it is difficult to control the depth and the advancement of the epidural needle firmly and reliably, especially when the epidural needle is inserted into the ligamentum flavum, even worse, if the ligamentum flavum is calcified. The depth of the epidural needle inserted is determined by calculating the difference of total length of the epidural needle and the length of the part of the epidural needle left outside the skin. The apparatus and method invented offer easy and reliable control over the depth and the advancement of the epidural needle during the insertion of the epidural needle into the epidural space and indicate directly the depth of the epidural needle having been inserted.

### BRIEF SUMMARY OF THE INVENTION

The object of the invention is to provide an apparatus and a method, which control and limit the depth and advancement of penetration of the epidural needle during the insertion of the epidural needle into the human epidural space for the epidural anesthesia or drug administration.

The apparatus of the invention includes an epidural needle holder with a slot to hold the epidural needle in its midline, a sliding plate with a sliding slot and two syringe holding walls to support the wing of the epidural needle and an attached syringe, and a screw on the side of the said sliding plate with a screw nut to adjust the limited depth and advancement of the epidural needle by blocking the sliding and advancing of the wing of the epidural needle over the said sliding plate. The invention further includes a scale in centimeter marked on each side of the said sliding plate to indicate directly the depth of the epidural needle having been inserted, and two finger holders with an elastic band for the anesthesiologist to hold the apparatus easily.

The invention differs from the prior art in that the depth and advancement of the epidural needle insertion is limited and fixed to half to one millimeter in each attempt of advancement during the insertion of the epidural needle into the epidural space through the multiple layers of the tissues. The said screw nut driven forwards and backwards in front of the wing of the epidural needle offers the space for the epidural needle with the syringe to advance further and deeper. The distance between the said screw nut and the wing of the epidural needle is the depth that the epidural needle can advance further, since the said screw nut blocks the wing of the epidural needle to slide and advance forwards. The said screw nut to block the wing of the epidural needle is used to prevent the sudden and too much advancement of the epidural needle in each attempt of insertion to avoid penetration of the dura. To advance the epidural needle deeper again, the said screw nut needs to be driven forwards half to one centimeter to offer a space for the epidural needle with the syringe to advance forwards half to one millimeter. This performance is repeated and the epidural needle is inserted deeper and deeper until the tip of the epidural needle into the epidural space is identified and confirmed with the loss of resistance technique, which is currently used in practice.

A scale in centimeter marked on each side of the said sliding plate indicates how many centimeters deep the tip of the epidural needle has been inserted from the skin.

### BRIEF DESCRIPTION OF FOUR VIEWS OF THE DRAWING

The embodiment of the invention is described in the following with reference to the accompanying drawing:

FIG. 1 is a perspective view of the depth controller for epidural needle.

FIG. 2 is a lateral plan view of the depth controller for epidural needle.

FIG. 3 is a top plan view of the depth controller for epidural needle.

FIG. 4 is a top plan view of the depth controller for epidural needle placed and used under an epidural needle with wing and an attached syringe.

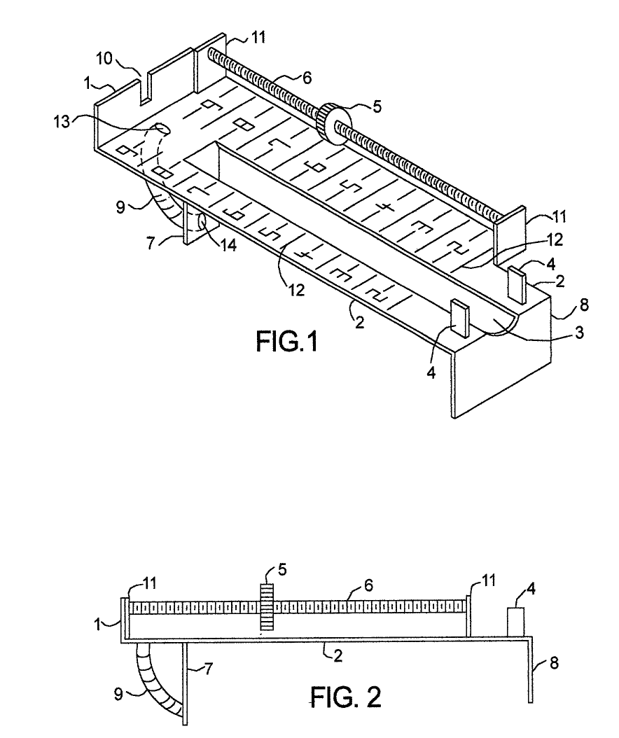

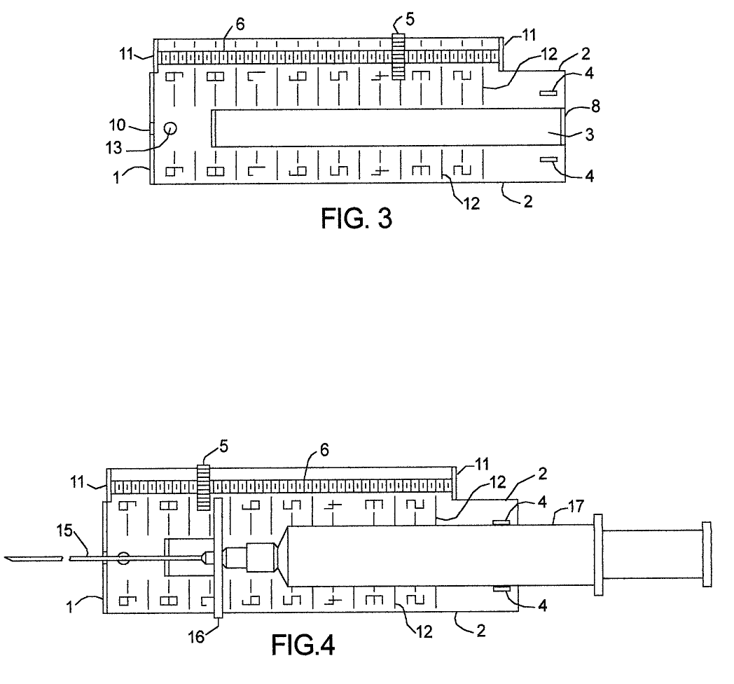

### DETAILED DESCRIPTION OF THE INVENTION

The depth controller of the invention comprises an epidural needle holder 1 with a needle slot 10, a sliding plate 2 with a sliding slot 3, two syringe holding walls 4, a screw nut 5 with screw bolt 6, a front finger hold 7, a rear finger hold 8, an elastic band 9 and a scale 12 marked on each side of the sliding plate 2.

The epidural needle holder 1 is a plate with a needle slot 10 in its middle. The width of the needle slot 10 is larger than the diameter of the epidural needle 15, so that the needle slot 10 holds the epidural needle 15 in the midline of the epidural needle holder 1 and the long axis of the sliding slot 3. The epidural needle holder 1 forms an angle with the sliding plate 2 and is placed against on the back of the patient to support the depth controller during the insertion of the epidural needle 15.

The sliding plate 2 is a support for the epidural needle's wing 16 to slide on when the epidural needle 15 is advanced. There is a scale 12 as a ruler in centimeter on each side of the sliding plate 2 to indicate directly the depth of the epidural needle 15 having been inserted.

There is a sliding slot 3 in the sliding plate 2. The width of the sliding slot 3 is a little smaller than the diameter of the syringe 17, so that the sliding slot 3 can hold the syringe 17. The sliding slot 3 and two syringe holding walls 4 support and hold the syringe 17 in the midline of the epidural needle holder 1, the needle slot 10 and the sliding slot 3 during the advancement of the epidural needle 15 into the epidural space. The syringe 17 slides over the edges of the sliding slot 3 during the advancement of the epidural needle 15.

The syringe holding walls 4 are two plates standing up at the rear part of the sliding plate 2. Two syringe holding walls 4 hold the syringe 17 with the epidural needle 15 in the midline of the sliding slot 3 and keep the syringe 17 from moving to either side of the sliding slot 3.

On one side of the sliding plate 2, there is a screw 5, 6 (screw nut 5, screw bolt 6) supported by two supporters 11, which stand up on one side of the sliding plate 2. The screw bolt 6 is parallel to the long axis of the sliding slot 3. The screw bolt 6 is positioned close to the outside edge of the epidural needle's wing 16. The epidural needle's wing 16 is advanced along the edge of the screw bolt 6 during the insertion of the epidural needle 15. The screw nut 5 blocks further advancement of the epidural needle 15 when the epidural needle's wing 16 hits the screw nut 5. As shown in the FIG. 4, the screw nut 5 needs to be turned and driven half to one millimeter further toward the patient to allow the epidural needle 15 to advance half to one millimeter further and deeper in each attempt.

The front finger hold 7 is a holding plate under the front part of the sliding plate 2 for the index finger of the anesthesiologist to hold the depth controller for epidural needle easily.

The rear finger hold 8 is a holding plate under the rear end of the sliding plate 2 for the anesthesiologist to use another hand to hold the depth controller for epidural needle easily.

There is a hole 13 at the lower end of the front finger hold 7. There is another hole 14 at the front end of the sliding plate 2. The two ends of the elastic band 9 are tied to the hole 13 and hole 14, respectively. The elastic band 9 ties the index finger of the anesthesiologist to the front finger hold 7, so that the anesthesiologist can hold the depth controller for epidural needle easily and tightly.

As shown in FIG. 4, to use the depth controller, the anesthesiologist holds the depth controller with the index finger on the front finger hold 7 with the elastic band 9, places the depth controller under the epidural needle 15 with the syringe 17, which has already been inserted into the interspinous ligament in the regular way in the current practice. The epidural needle holder 1 and the index finger are placed and supported on the back of the patient. The epidural needle 15 is positioned in the needle slot 10 of the epidural needle holder 1. The syringe 17 is positioned on the sliding slot 3 of the sliding plate 2 and between two syringe holding walls 4. The epidural needle's wing 16 is positioned on the sliding plate 2. The screw nut 5 is turned and driven half to one millimeter in front of the epidural needle's wing 16. The thumb of the non-injecting hand holds the epidural needle's wing 16 to push and advance the epidural needle 15. Alternatively, the thumb of the injecting hand holds the rear part of the syringe 17 to push and advance the epidural needle 15, with the index finger holding on the rear finger hold 8. The screw nut 5 blocks further advancement of the epidural needle 15 when the epidural needle's wing 16 hits the screw nut 5. The screw nut 5 needs to be turned and driven half to one millimeter further toward the patient to allow the epidural needle 15 to advance half to one millimeter further and deeper in each attempt until entering the epidural space, which is identified and confirmed with the lose of resistance technique, with multiple attempts. An epidural catheter can be placed or a drug can be administered into the epidural space through the epidural needle.

We claim the following:

1. An apparatus for holding an epidural needle together with a syringe and controlling the depth and the advancement of penetration of the epidural needle during insertion of the epidural needle into the human epidural space, comprising:
a. a plate-like epidural needle holder with a needle slot in its middle for holding the epidural needle in the said slot in the midline of the said epidural needle holder and for supporting the said apparatus on the back of the patient.
b. a needle slot, which is a little wider than the diameter of the epidural needle, in the middle of the said epidural needle holder for holding the epidural needle in the midline of the said epidural needle holder.
c. a sliding plate, which forms an angle with the said epidural needle holder, for supporting the wing of the epidural needle, the syringe, two supporters of a screw and two syringe holding walls.
d. two syringe holding walls on the rear part of the said sliding plate for keeping the syringe from moving from side to side.
e. a screw with two supporters on the side of the said sliding plate for controlling the depth and the advancement of the epidural needle with a screw nut to block the wing of the epidural needle to advance forwards over the said sliding plate.
f. a front finger hold below the front part of the said sliding plate for the index finger to hold the said apparatus easily.
g. a rear finger hold below the rear part of the said sliding plate for another index finger to hold the said apparatus easily.
h. an elastic band connected between two holes in the lower end of the said front finger hold and the front end of the said sliding plate, respectively, for the index finger to hold tightly and easily the front finger hold.
i. a scale in centimeter marked on each side of the said sliding plate for indicating directly how many centimeters deep the tip of the epidural needle has been inserted from the skin.

2. A method for holding an epidural needle together with a syringe and controlling the depth and the advancement of penetration of the epidural needle during insertion of the epidural needle into the human epidural space, comprising the following steps:
a. inserting an epidural needle into the interspinous ligament between two spinous processes of the human vertebrae through the skin, subcutaneous tissue and supraspinous ligament as the regular way after the preparation, drape and application of the local anesthetic with the sterile technique.
b. removing the stylet from the epidural needle and connecting a syringe filled with some air or saline to the epidural needle.
c. holding the said apparatus with the index finger over the said front finger hold with the said elastic band.
d. placing the said apparatus under the epidural needle and syringe with the said epidural needle holder and the index finger supported on the patient's back with the epidural needle in the said needle slot of the said epidural needle holder and with the syringe over the said sliding slot of the said sliding plate and between two said syringe holding walls, and the wing of the epidural needle on the said sliding plate.
e. turning and driving the said screw nut half to one millimeter in front of the wing of the epidural needle and offering half to one millimeter of space for the wing of the epidural needle together with the epidural needle and the syringe to advance further and deeper.
f. advancing the epidural needle further and deeper until the said screw nut blocks the wing of the epidural needle to advance further.
g. using the lose of resistance technique to identify and confirmn the tip of the epidural needle in the epidural space.
h. repeating the steps of e, f and g until the tip of the epidural needle in the epidural space is identified and confirmed with the lose of resistance technique.
i. placing an epidural catheter or administering a drug into the epidural space through the epidural needle as the regular way.
